(19) United States
(12) Patent Application Publication
(10) Pub. No.: US 2012/0067355 A1
(43) Pub. Date: Mar. 22, 2012

(54) SURGICAL DRAPE TO FACILITATE EXTREMITY SURGERY

(76) Inventor: Amir A. Jamali, Sacramento, CA (US)

(21) Appl. No.: 13/239,172

(22) Filed: Sep. 21, 2011

Related U.S. Application Data
(60) Provisional application No. 61/403,720, filed on Sep. 21, 2010.

(51) Int. Cl. A61B 19/12

(52) U.S. Cl. 128/856

(57) ABSTRACT

A device and method for the performance of lower extremity surgery through the use of a bilaterally fenestrated drape with an operative side having an open fenestration and a nonoperative side having an extremity tube sealed over a fenestration, the extremity tube being closed at one end.

### CROSS REFERENCES TO RELATED APPLICATIONS

The present application claims the benefit of U.S. Provisional Patent Application Ser. No. 61/403,720 filed Sep. 21, 2010 (Sep. 21, 2010.)

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention relates a device and technique used for draping of a surgical extremity at the time of surgery in such a way to allow isolation of the contralateral extremity.

2. Discussion of Related Art Including Information Disclosed under 37 CFR §§1.97, 1.98

The field of orthopedic surgery often requires the use of fluoroscopy. However working on the extremities is associated with interference from the contralateral extremity. A method is required to mobilize the contralateral extremity out of the field of view of the radiographic source. One potential strategy is to place the contralateral leg in a well leg holder. This is not available on all surgical tables. Another option is to prepare and to drape both extremities. This can be associated with increased surgical time and higher rate of contamination. A specialized tool or drape is required that would allow complete mobilization of the contralateral non-injured and non-operative extremity while not requiring inclusion of that extremity in the surgical field.

### BRIEF SUMMARY OF THE INVENTION

The present invention is a surgical drape system, and a method for using the same, which allows the contralateral, non-surgical extremity to be fully mobile during extremity surgery in order to facilitate positioning of the operative extremity under the nonsurgical extremity. The surgical drape consists of a bilaterally fenestrated surgical drape with a separate extremity tube sealed over the fenestration for the nonoperative leg. The placement of the nonoperative leg into the extremity tube allows complete mobilization of this extremity at the hip. This mobilization facilitates the positioning of the operative leg in such a way to allow optimal radiographic views with a fluoroscopy unit as in the case of treatment of hip fractures, femoral fractures, or tibial fractures. Additionally, this mobilization allows for the placement of the operative leg under the nonoperative leg, facilitating procedures such as total hip arthroplasty through the direct anterior approach.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 is an upper lateral perspective view of the surgical drape;

FIG. 2 is an upper anterior perspective view of the surgical drape;

FIG. 3 is an upper lateral view of the patient with both legs elevated and flexed at the hip;

FIG. 4 is an upper lateral view of the patient with partial placement of the surgical drape;

FIG. 5 is an upper lateral view of the patient with final placement of the fenestrations at the level of the hip;

FIG. 6 is an upper lateral view of the patient after final placement of the drape over the patient torso; and

FIG. 7 is an upper lateral view of the patient after final placement of the drape with the operative hip and operative leg in a figure of four position.

### DETAILED DESCRIPTION OF THE INVENTION

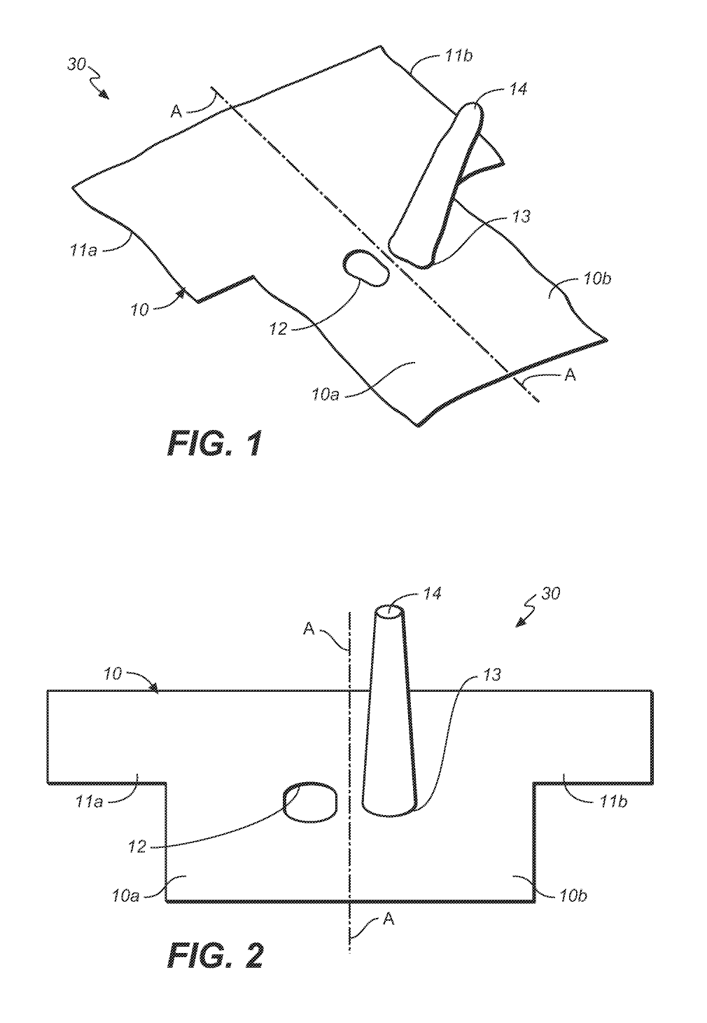

Referring to FIGS. 1 through 7, wherein like reference numerals refer to like components in the various views, there is illustrated therein a new and improved surgical drape, generally denominated 30 herein.

FIG. 1 is an upper lateral perspective view of the surgical drape 30 showing the body 10 of the drape divided along an axis A into an operative side 10a and a non-operative side 10b. The drape body 10 is preferably made from a fluid impermeable medical fabric panel (e.g., paper, plastic, or fabric) and may include laterally extending wings 11a, 11b, though a simple rectangular panel is also suitable. The operative side 10a of the drape 30 includes an operative side fenestration 12, while the non-operative side 10b includes a nonoperative side fenestration 13 with a sealed and attached extremity drape (or extremity tube portion) 14, preferably attached to the edges of the fenestration opening through a sonic sealing process rather than stitching, though any suitable attachment process is contemplated. The fenestrations are each preferably circular in shape and sized between 10 cm and 50 cm in diameter.

FIG. 2 is an upper anterior perspective view of the surgical drape 30 demonstrating the body of the drape 10, the operative side fenestration 12, and the nonoperative side fenestration 13 with the sealed and attached extremity drape 14.

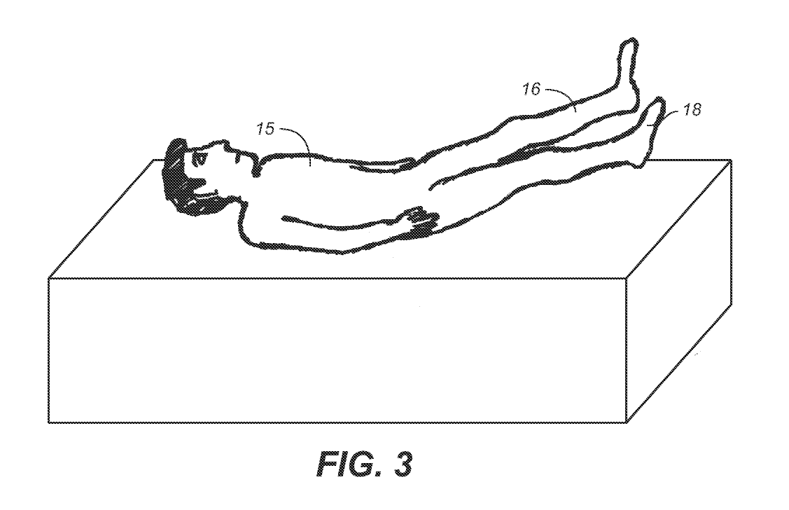

FIG. 3 is an upper lateral view of the patient 15 being prepared for right hip direct anterior total hip replacement with both legs elevated and flexed at the hip on the operative table. The nonoperative leg 16 and the operative leg 18 are shown.

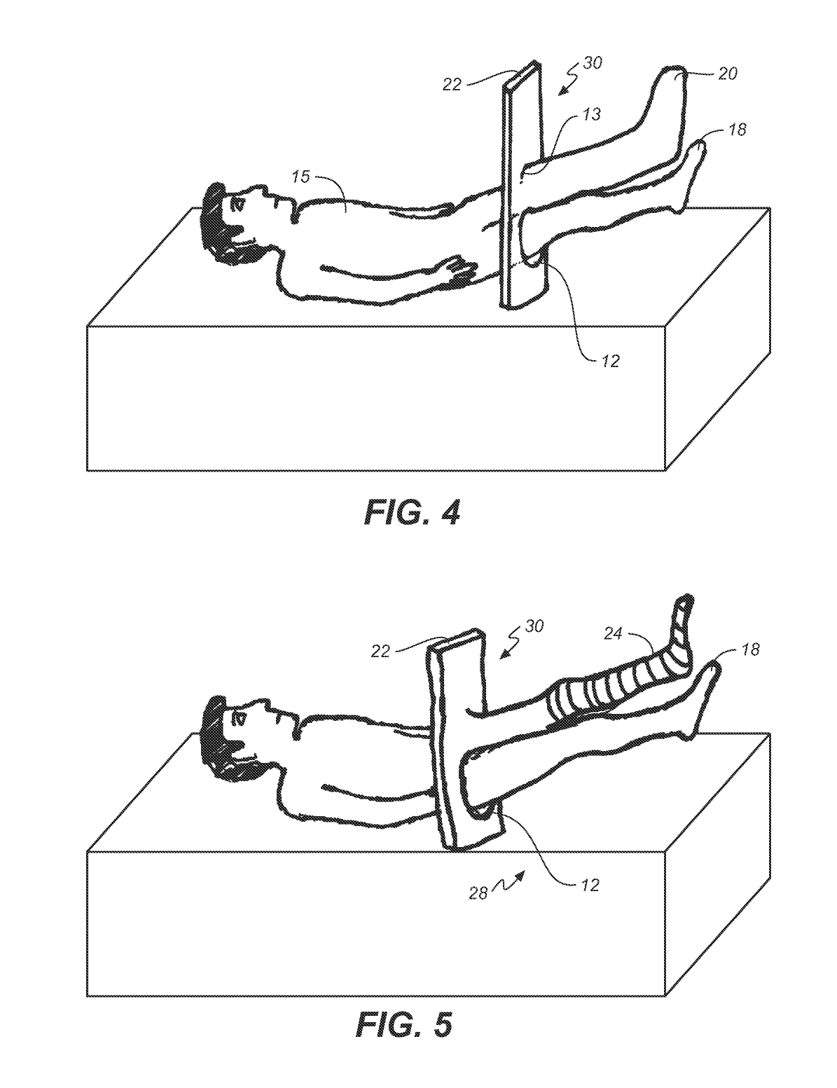

FIG. 4 is an upper lateral view of the patient of FIG. 3 with his or her non-operative leg passed through the non-operative leg fenestration 13 and the extremity tube 20 sealed to the nonoperative leg fenestration of the surgical drape 30, shown here schematically as only a portion 22 of the drape material so as to reveal and make clear the process of placing the drape over the patient. The operative leg 18 is seen as passed through the open fenestration 12 on the operative side of the drape.

FIG. 5 is an upper lateral view of the patient showing the final placement of the extremity tube to encompass the nonoperative leg and passage of the (partially shown 22) surgical drape 30 in its final position at the level of the groin. The extremity tube is then stabilized around the nonoperative leg using standard bandage material 24. The operative leg 18 is seen as passed through the open fenestration 12 on the operative side 28 of the surgical drape 30.

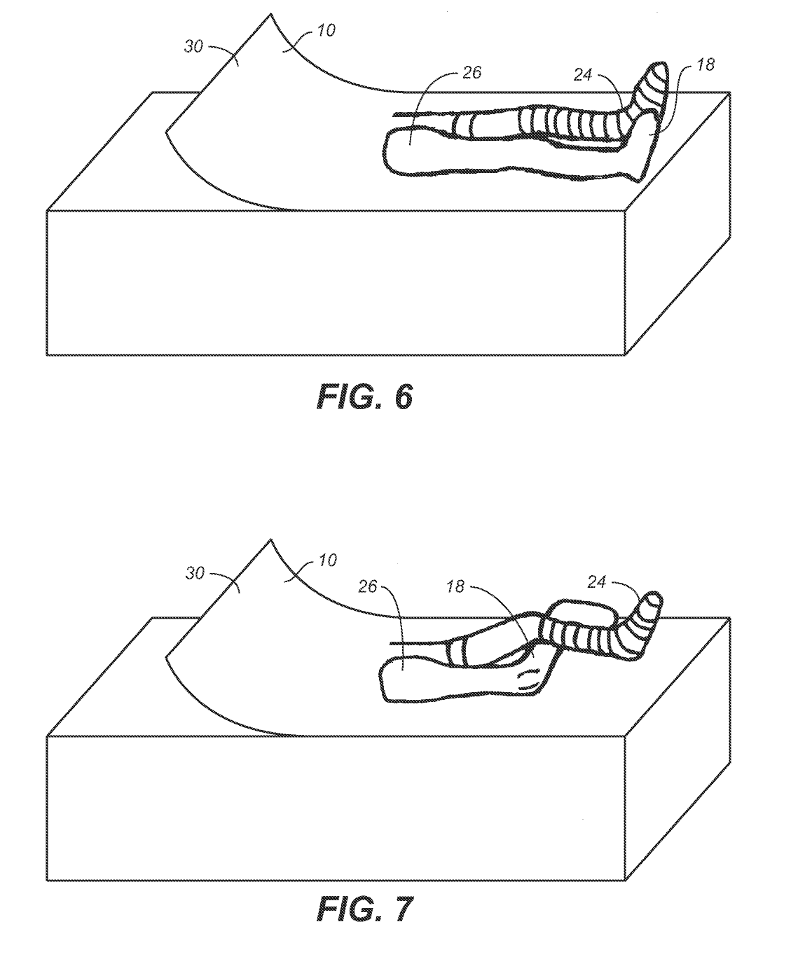

FIG. 6 is an upper lateral view of the patient after final placement of the drape 22 over the patient's torso. The operative hip 26 and operative leg 18 are now fully accessible. The nonoperative leg can be moved toward the ceiling to allow placement of the operative leg under the nonoperative leg which is sealed within the extremity tube and bandages 24.

FIG. 7 is an upper lateral view of the patient after final placement of the drape 22 with placement of the operative hip 26 and operative leg 18 in the “Figure of Four” position, under the nonoperative leg which is sealed within the extremity tube and bandages 24.

The above disclosure is sufficient to enable one of ordinary skill in the art to practice the invention, and provides the best mode of practicing the invention presently contemplated by the inventor. The full and complete disclosure of the preferred embodiments of this invention does not limit the invention to the exact construction, dimensional relationships, and operation shown and described, inasmuch as modifications, alternative constructions, changes and equivalents will readily occur to those skilled in the art and may be employed, as suitable, without departing from the true spirit and scope of the invention. Accordingly, the above description and illustrations should not be construed as limiting the scope of the invention, which is defined by the appended claims.

What is claimed as invention is:

1. A surgical drape for lower extremity surgery, comprising:
a drape body divided bilaterally into an operative side and a non-operative side;
an open fenestration disposed on said operative side;
a nonoperative side fenestration disposed on said non-operative side;
an extremity tube having a closed first end and an open second end, said second end sealed to said nonoperative side fenestration.

2. The surgical drape of claim 1, wherein said drape body and said extremity tube are fabricated from fluid impermeable plastic, paper, or fabric.

3. The surgical drape of claim 1, wherein said operative side fenestration and said non-operative side fenestration each measure between 10 cm and 50 cm in diameter.

4. A surgical drape having an open operative fenestration and a second nonoperative fenestration having an extremity tube sealed over said second fenestration, wherein during drape passage, a nonoperative extremity of the patient is passed into said extremity tube while the patient's operative extremity is brought forth through said open operative fenestration.

5. A surgical drape for use in connection with lower extremity surgery, including total hip replacement, said surgical drape comprising a drape body divided bilaterally to include an open fenestration and a sealed fenestration, said sealed fenestration having an extremity tube disposed over the fenestration opening, such that the extremity draped by the extremity tube can be mobilized freely to allow for intraoperative positioning to facilitate surgical procedures.
